(19) United States
(12) Patent Application Publication
(10) Pub. No.: US 2026/0275261 A1
(43) Pub. Date: Sep. 17, 2026

(54) Cell tissue culture and transport container

(71) Applicant: ADHARA, Paris (FR)

(72) Inventors: Ronan Garo, Dijon (FR); Mérouane Alliouche, Dijon (FR); Pascal Valois, Dijon (FR); Pierre-Noël Lirsac, Strasbourg (FR); Mathilde Girard, Chenove (FR)

(73) Assignee: ADHARA, Paris (FR)

(21) Appl. No.: 18/993,424

(22) PCT Filed: Jul. 10, 2023
(86) PCT No.: PCT/FR2023/051065

§ 371 Date: Jan. 10, 2025

(30) Foreign Application Priority Data
Jul. 13, 2022 (FR) FR2207247

(51) Int. Cl. C12M 1/00; C12M 1/12; C12M 3/00

(52) U.S. Cl. CPC C12M 23/52; C12M 23/22; C12M 23/28; C12M 23/38; C12M 23/42; C12M 25/00; C12M 29/04; C12M 37/02; C12M 37/04

(57) ABSTRACT

The invention relates to a cell tissue culture and transport canister comprising: a canister body having an accessible interior and an upper opening; a filter arranged inside the canister body or outside the canister and placing the interior of the canister body in communication with an exterior of the canister body, the filter being configured to allow a gas exchange between the interior and the exterior of the canister body, while preventing contamination of the cell tissue culture; a closure means closing the upper opening of the canister body, the closure means being sealed or slightly permeable with respect to fluids and gases; and at least one aseptic fluid connection through the canister body, the at least one aseptic fluid connection including two fluid inlet connections and one fluid outlet connection.

### TECHNICAL FIELD

The present disclosure relates to cell tissue culture and transport containers.

### PRIOR ART

The process of manufacturing cell tissue, such as artificial skin, involves the use of a sterile environment and a plurality of culture media brought into culture sequentially. The process may be time-consuming and involve a lot of manipulation of the closed system.

Typically, stem cells are placed in a culture container. This culture container is a closed environment in which the different layers of skin will be created by adding culture media. Each time a medium is to be supplied to the culture, the operator uncaps/unscrews/opens the container and introduces the culture medium. They then re-cap/re-screw/close the container, put it back and let it rest. They take the container, uncap it, insert and re-cap it for each culture medium.

The multiple operations involved in opening and closing the container can lead to contamination of the culture medium. This also requires a large amount of operator time, which does not allow for large-scale multiplication of the cell tissue culture.

### SUMMARY

A cell tissue culture and transport container is proposed comprising: a container body having an accessible interior and an upper opening allowing communication between the interior and an exterior of the container body; a filter arranged in the container body or on the exterior of the container body, the filter placing the interior in communication with the exterior of the container body, the filter being configured to allow a gas exchange between the interior and the exterior of the container body, while preventing contamination of the cell tissue culture; a closure means closing the upper opening of the container body, the closure means being sealed or slightly permeable with respect to fluids and gases; and at least one aseptic fluidic connection through the container body, said at least one aseptic fluidic connection including two fluidic inlet connections for fluids and one fluidic outlet connection for fluids.

The features disclosed in the following paragraphs may optionally be implemented independently of one another or in combination with one another:
the closure means is disposable, such that once it has been removed from the upper opening, the interior of the container body is accessible through the upper opening
the closure means is a cover or a seal
the closure means is sealed, screwed or clipped to the container body, e.g., by means of an O-ring, heat sealing or ultrasonics
the closure means is transparent
the closure means retains its mechanical properties down to an exposure temperature of approx. −200° C.
the closure means is made of polyethylene terephthalate or polyethylene
the filter is arranged in the region of said at least one fluidic connection
the filter is higher than the culture medium to ensure its gas exchange function and not be wetted by the culture medium
the filter is arranged in a horizontal part or in an inclined part of an interior of the container body-the inclined part being inclined toward the insert
the filter is arranged vertically below a gripping area of the insert
the filter is arranged in the container body distal to a culture area of the insert
the filter is made of polyethylene, polyurethane, polyester, or polypropylene
the filter is hydrophobic, preferably on its two surfaces
the container also comprises a removable insert designed to be arranged in the interior of the container body, the insert being designed to receive a cell tissue culture
the insert can be removed from the container body through the upper opening
the insert has a culture area and a gripping area connected to the culture area, the gripping area being designed to manipulate the insert relative to the container body
the culture area includes an upper opening designed to accommodate the cell tissue culture
the culture area includes a porous base
the porous base faces the upper opening when the insert is arranged in the container body
the porous base of the insert is made of polyester, polyethylene, or polycarbonate
the size of the pores of the porous base is 0.2 to 4 μm, preferably 0.2 to 0.6 μm
the density of the pores of the porous base is 1*10⁶ to 5*10⁶, preferably 1*10⁶ to 3*10⁶ pores per cm²
the gripping area is inclined toward the culture area
the culture area of the insert is rounded
the culture area of the insert is a ring
the culture area of the insert has a thickness corresponding to a thickness of a working surface of the container body designed to receive the culture area of the insert
the culture area of the insert has a shape corresponding to a shape of a working surface of the container body designed to receive the culture area of the insert
said insert is resistant to dimethyl sulfoxide (DMSO)
the insert includes at least one connection designed to cooperate in a removable manner with the interior of the container body
said at least one connection is at least one tab designed to be removably inserted into a housing on the interior of the container body
the housing is an open recess
the insert is made of polyester, polypropylene, fluorinated ethylene propylene, or polycarbonate
the interior of the container body includes a working area designed to receive the cell tissue culture and fluid culture media allowing the cell tissue to grow
the working area includes a working surface designed to receive the culture area of the insert
the working surface preferably being flat
the working surface has undergone a plasma surface treatment or a corona treatment
the interior of the container body includes an inclined part directed toward the working surface
the working surface is transparent
the container body is substantially parallelepipedic and has rounded edges
the working surface can be removed from the rest of the container body
the container body is made of polyester, polypropylene, fluorinated ethylene propylene, polycarbonate, polycarbonate alloy, or Eastman Tritan™ Copolyester MP100
the two fluidic inlet connections for fluids are arranged vertically above the culture area
the fluidic outlet connection for fluids is arranged vertically at the same level as or below the culture area
the two fluidic inlet connections for fluids are ports having a tubular extension extending toward the interior of the container body
the tubular extension has an end vertically above the insert
one end of the fluidic inlet connections in the interior of the container body is beveled
the bevel is oriented toward the upper opening
the bevel is approximately 45 degrees
the tubular extension is sealed to the container body by welding, and the bevels are obtained using an aseptic disconnection machine
the fluidic outlet connection for fluids is a septum, a hose clamp, a barbed fitting with a radial crimping system, or a Luer Lock conical fitting
said at least one fluidic connection is resistant to dimethyl sulfoxide (DMSO)
the container body also comprises a partition separating the working area from the filter.

### BRIEF DESCRIPTION OF THE DRAWINGS

Further features, details and advantages will become apparent upon reading the detailed description below and upon examining the attached drawings, in which:

FIG. 1 is a schematic perspective view from above of a cell tissue culture and transport container according to one embodiment;

FIG. 2 is a schematic perspective view from below of the container from FIG. 1;

FIG. 3 is another schematic perspective view from above of the container from FIG. 1;

FIG. 4 is a schematic sectional view of the container from FIG. 1;

FIG. 5 is a schematic perspective view of an insert for the container from FIG. 1; and

FIG. 6 is a schematic view of an embodiment of the insert in which a porous base of the insert is detachable;

FIG. 7 is a schematic view of another embodiment of the insert in which a porous base of the insert is detachable;

FIG. 8 is a schematic view of an embodiment of the container in which the container has a removable base; and

FIG. 9 is a schematic view of another embodiment of the container in which a filter is arranged vertically;

FIG. 10 is a schematic view of another embodiment of the container which includes a partition between the filter and the working area.

### DESCRIPTION OF THE EMBODIMENTS

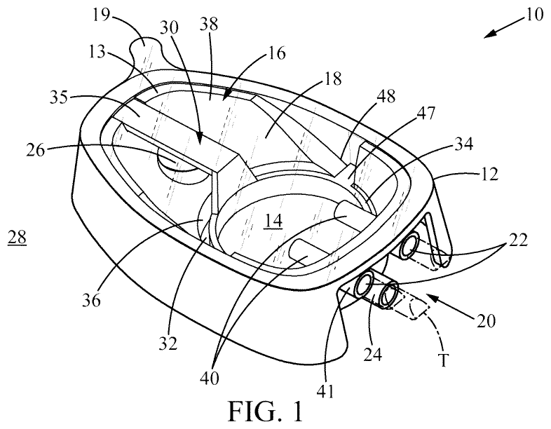

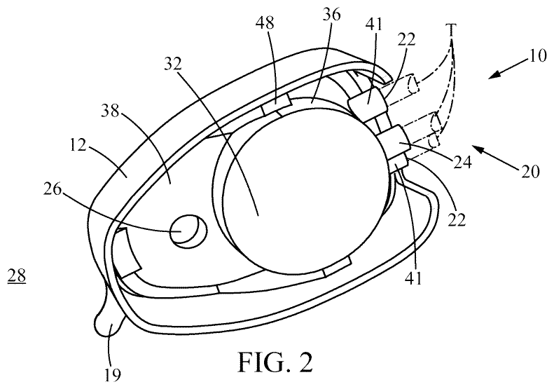

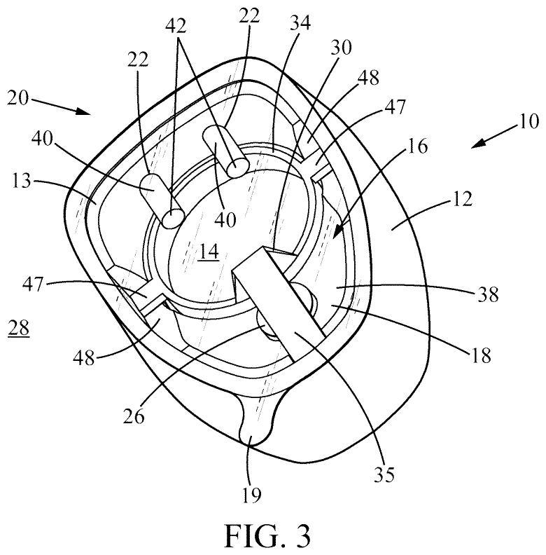

A cell tissue TC culture and transport container 10 according to one embodiment will be described with reference to FIGS. 1 to 4. The container 10 comprises a container body 12 having an accessible interior 14 and an upper opening 16. The upper opening 16 is closed by a closure means 18. The closure means 18 is preferably sealed with respect to fluids and gases. According to another embodiment, the filter is slightly permeable with respect to fluids and gases. A filter is slightly permeable if the water vapor transmission rate is less than 5 g/m²/24 h, preferably 1 g/m²/24 h.

The container 10 comprises at least one aseptic fluidic connection 20 through the container body 12. In the example from FIG. 1, the container 10 includes three aseptic fluidic connections 20: two fluidic inlet connections 22 for culture fluids, and one fluidic outlet connection 24 for fluids. The fluidic inlet connections 22 allow fluids, preferably liquids, to be conveyed, enabling the cell tissue TC to be cultured, while the fluidic outlet connection 24 allows the cell tissue TC culture to be drained of these same fluids. The interior 14 of the container body 12 includes a working area (or volume) 36 designed to receive the cell tissue TC culture and the fluid culture media allowing the cell tissue TC to grow.

The container 10 also includes a filter 26 arranged in the container body 12 (i.e., in the wall delimiting the interior from the exterior of the container 10). The filter 26 places the interior 14 of the container body 12 in communication with an exterior 28 of the container body 12. The filter 26 is configured to allow a gas exchange between the interior 14 and the exterior 28 of the container body 21, while preventing the passage of liquids. The filter 26 is configured to prevent the passage of bacteria and viruses. The filter 26 is the only means of communication between the interior and the exterior of the container 10, apart from three aseptic fluidic connections 20.

According to one embodiment, the filter 26 is not arranged in the container body 12 but on the exterior 28 of the container body 12. It would communicate with the interior 14 of the container body 12 via a connecting means, such as a tube connected to the container body 12. Positioning the filter on the exterior 28 of the container body 12 makes it possible to prevent pressure deltas (of the order of −0.5 bar) during cryopreservation.

According to one embodiment, the filter is hydrophobic. According to one embodiment, a hydrophobic treatment has been applied to each surface of the filter.

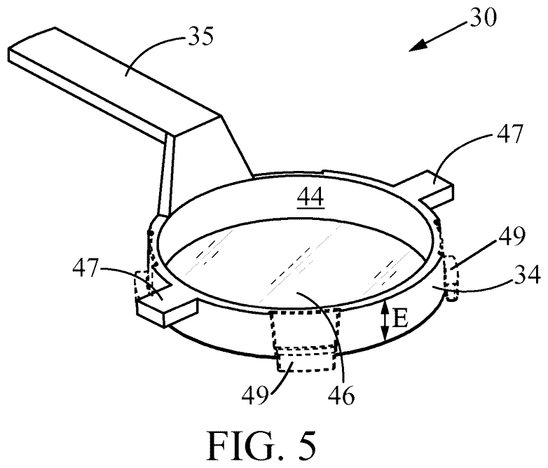

The container 10 may optionally include a removable insert 30 arranged in the working area 36 and designed to receive the cell tissue TC culture. The insert 30 may be inserted into the interior 14 of the container body 12 through the upper opening 16. As a result, the upper opening 16 is sized to allow the insert 30 to pass through. The insert 30 may have a culture area 34 designed to receive and culture the cell tissue TC and a gripping area 35 for manipulating the insert 30, in particular for inserting it into and/or removing it from the container body 12. The insert 30 will be described in more detail in the following, with reference to FIG. 5. According to other embodiments, the container 10 could not have an insert, such that the cell tissue TC culture is carried out directly on the interior 14 of the container body 12 in the working area 36.

The working area 36 is sized to contain an adequate volume of culture liquid. In the case of the insert 30, the working area 36 has a volume slightly greater than that of the culture area 34 of the insert 30.

The working area 36 of the container body 12 includes a working surface 32 designed to receive the cell tissue TC culture. In the case of containers having an insert, the working surface 32 is designed to receive the culture area 34 of the insert 30. In the case of containers 10 without an insert, the cell tissue TC will be arranged directly on the working surface 32 of the container 10. In this latter case, the working surface 32 may have undergone a plasma surface treatment to enable the cell tissue TC to cling to the working surface 32. Alternatively, the working surface 32 may have undergone a corona treatment.

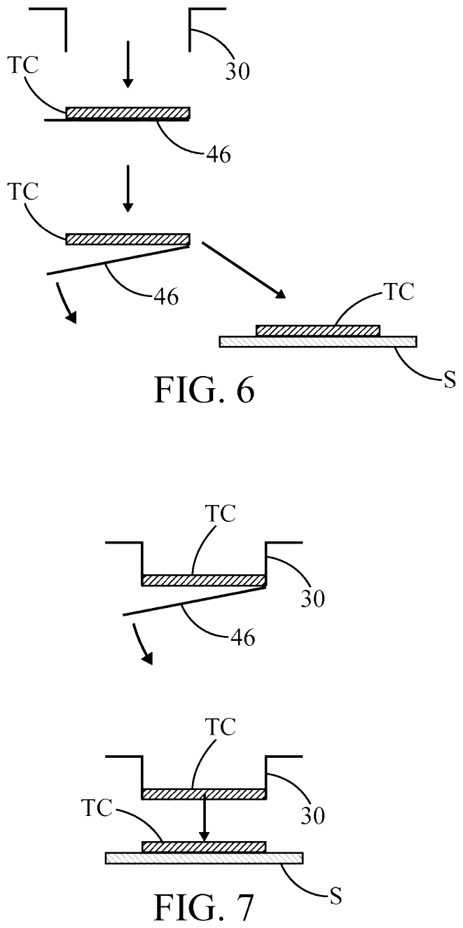

Whether with or without an insert, the working surface 32 can, for example, be transparent to allow visual inspection of the cell tissue TC culture, without having to open the closure means 18. The working surface 32 may form part of the container body 12 delimiting the interior 14 from the exterior 28. More generally, all or part of the container body 12 could be transparent. The working surface 32 is preferably flat. It can be a reinforcement of the container body 12, as shown in the figures. The working surface 32 may be detachable from the container body 12 in order to extract the cell tissue TC when it is formed, as will be explained in relation to FIGS. 6 to 8.

The closure means 18 is used to create a closed system in the container 10, enabling the cell tissue TC to be cultured. The closure means 18 may be sealed to the container body 12, for example by heat sealing or ultrasonics. The closure means 18 may include a tongue 19 which allows the closure means 18 to be removed by hand without the need for instruments, such as a scalpel.

The closure means 18 may be disposable, such that once it has been removed from the upper opening 16, the interior 14 of the container body 12 is accessible through the upper opening 16. In this way, the cell tissue culture and transport container 10 may be a disposable product. In the example of the figures, the closure means 18 is transparent in order to allow visual inspection of the cell tissue culture. The closure means 18 may not be transparent, or may be partially transparent, colored or clear. In order to withstand cryogenic freezing during transport and storage of the cell tissue culture, the closure means 18 is preferably designed to retain its mechanical properties down to an exposure temperature of approximately −200° C. The closure means 18 is made of polyethylene terephthalate or polyethylene, for example.

The container body 12 may be made by plastic injection molding. The container body 12 may be made of polypropylene, fluorinated ethylene propylene, polycarbonate, or Eastman Tritan™ Copolyester MP100, for example. The container body 12 may be designed to retain its mechanical properties down to an exposure temperature of approximately −200° C., to enable cryogenic freezing of the container 10. The container body 12 may be resistant to dimethyl sulfoxide (DMSO). The container body 12 may be substantially parallelepipedic with rounded edges in order to facilitate transport without loss of space. The container body 12 may have a generally ovoid shape when viewed from above.

The container body 12 may include a reinforcement 13 for receiving one end of the gripping area 35 of the insert 30, such that the insert 30 does not protrude from the container 10.

The interior 14 of the container body 12 may include an inclined part 38 directed toward the working surface 32. The inclined part 38 may allow fluids to flow toward the cell tissue TC culture area 36. The inclination of the inclined part 38 is relative to a horizontal plane H, the horizontal plane H containing the working surface 32. In one embodiment, the inclined part 38 faces the fluidic connections 20.

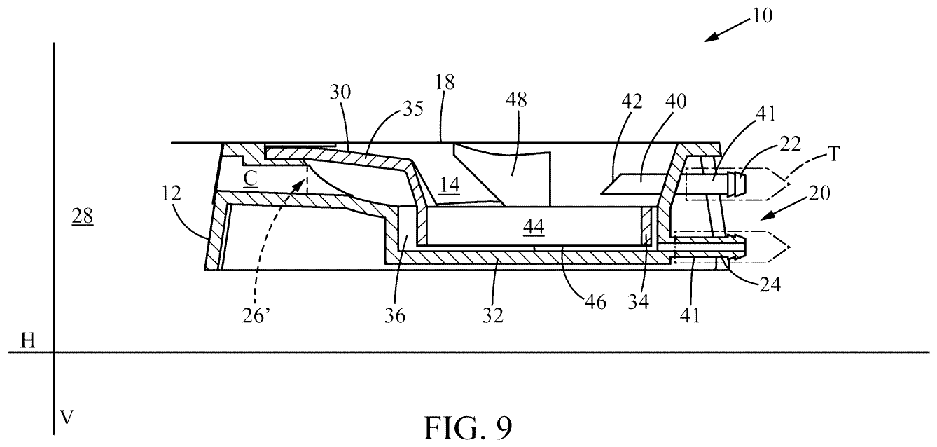

The inclined part 38 may receive the filter 26. The filter 26 may be arranged horizontally in the inclined part 38 or may even be inclined, for example with the same inclination as the inclined part, as shown in FIGS. 1 to 4. The filter 26 may be arranged vertically in the inclined part 38 as shown by the filter 26′ in FIG. 9. An evaluation channel C could then extend from the inclined part 38 through the filter 26′ and out of the container. According to another embodiment, the filter 26 is arranged in the region of the fluidic connections 20. The filter 26 may be arranged in the container body 12 distally to the working surface 32. This limits the risk of the filter 26 becoming wet. According to one embodiment, and as shown in the figures, the filter 26 may be arranged vertically below the gripping area 35 of the insert 30. The filter 26 may be is made of polyethylene, polyurethane, polyester, or polypropylene. In order to withstand cryogenic freezing during transport and storage of the cell tissue TC culture, the filter 26 is preferably designed to retain its mechanical properties down to an exposure temperature of approximately −200° C. The container 10 may include more than one filter.

According to another embodiment, the filter 26 is arranged in the container body 12 in the region of the fluidic connections 20. According to one embodiment, the filter 26 is arranged on the container body 12 opposite the fluidic connections 20. This latter configuration may limit the risk of the filter 26 becoming wet.

The two fluidic inlet connections 22 for fluids are ports having an internal tubular extension 40 extending toward the interior 14 of the container body 12. Each tubular extension 40 has an end lying vertically above the working surface 32, or if the container has an insert 30, vertically above the culture area 34 of the insert 30. The internal tubular extensions 40 are dimensioned to be long enough to supply the culture medium, but at the same time not so long as to impede removal of the insert 30. According to one embodiment, an end 42 of the fluidic inlet connections 22 in the interior 14 of the container body 12 is beveled. The beveled shape allows flow toward the culture area 34/working surface 32. For example, the bevel is oriented toward the upper opening 16. The bevel may be approximately 45 degrees. The internal tubular extensions 40 may be sealed to the container body 12 by welding and the bevels obtained using an aseptic disconnection machine.

The fluidic connections 20 also each have an external tubular extension 41 extending from the exterior of the container body 12 such as to be connected to tubes T containing culture or drainage liquids (shown as dotted lines). The external tubular extensions 41 are designed to connect to the tubes T, for example via a male-female system with a Serflex element which provides sealing, a mechanical system, by ultrasonic welding, or using biocompatible glue.

The fluidic inlet connections 22 and/or the fluidic outlet connection 24 for fluids may be a septum that can be pierced by a needle while ensuring sealing. The fluidic inlet connections 22 and/or the fluidic outlet connection 24 for fluids may be resistant to dimethyl sulfoxide (DMSO) or any other preservative medium injected into the container at the end of the cell culture process.

The container 10 could also have external fixings, for example a slot in the exterior of the container body 12, to enable the container to be temporarily fixed to a support allowing the supply of culture media to the container. The support can thus contain a plurality of containers 10 for parallel production of cell tissue TC.

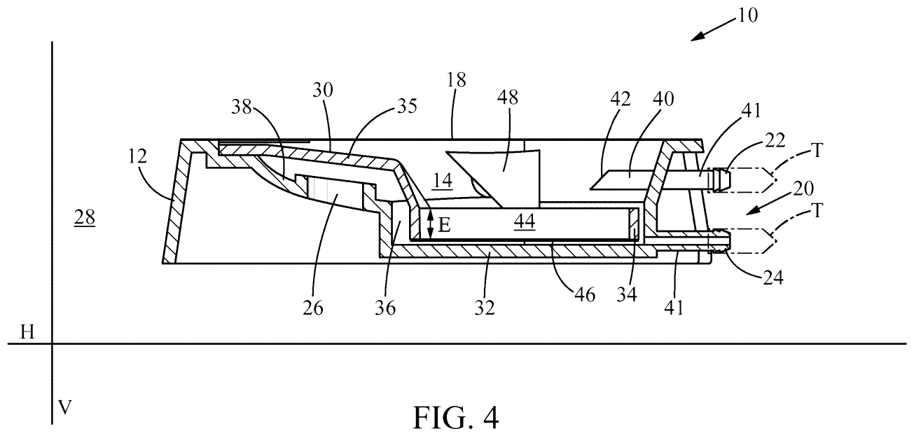

As mentioned above, the removable insert 30 comprises the culture area 34 designed to receive and culture the cell tissue TC and the gripping area 35 for manipulating the insert 30, in particular for inserting it into and/or removing it from the container body 12. This manipulation can be performed with the fingertips, or if the container 10 allows it (in particular due to the size of the cavity located under the gripping area 35 and visible in FIG. 4 through the inclined part 38) with an edge of the phalanges.

The insert 30 (culture area 34 and gripping area 35) may be made of polyester, polypropylene, fluorinated ethylene propylene, or polycarbonate.

The culture area 34 may include an upper opening 44 designed to accommodate the cell tissue TC culture in order to feed the cells from above, in particular via the fluidic inlet connections 22. The culture area 34 includes a porous base 46 facing the upper opening 16 when the insert 30 is arranged in the container body 12 on the working surface 32. The porous base 46 of the insert 30 makes it possible to receive the cell tissue TC culture. It is porous to allow the fluid culture medium to soak the top and bottom of the cell tissue TC culture. The porous base 46 may be made of polyethylene or polycarbonate. The porous base 46 may be transparent. The porous base 46 may be flexible or rigid. It may have undergone a plasma treatment. Porous is understood to mean that the base allows liquids to evacuate, either through a network of open cells or one or more evacuation holes. The porous base 46 may or may not be detachable from the rest of the insert 30. According to one embodiment, the size of the pores of the porous base is 0.2 to 4 μm, preferably 0.2 to 0.6 μm. According to one embodiment, the density of the pores of the porous base is 1*10⁶ to 5*10⁶, preferably 1*10⁶ to 3*10⁶ pores per cm².

The culture area 34 of the insert 30 is generally rounded, for example annular. The culture area 34 of the insert 30 may have a thickness E less than or corresponding to a thickness of the working surface 38 of the container body 12 designed to receive the culture area 34 of the insert 30. This can allow fluids to flow smoothly. According to one embodiment, the culture area 34 of the insert 30 has a shape corresponding to a shape of the working surface 32 of the container body 12 designed to receive the culture area 34 of the insert 30 to prevent the insert 30 from moving relative to the container body 12 during transport.

According to one embodiment and as shown in the figures, the gripping area 34 is a handle. However, the gripping area 34 could have any shape that allows the insert 30 to be manipulated. In the embodiment shown in the figures, the gripping area 34 is arranged opposite the inclined part 38. The gripping area 34 may be inclined with respect to the horizontal plane H toward the culture surface 32 to allow the flow of condensed fluids, for example. The inclination of the gripping area 34 with respect to the horizontal plane H may substantially be the inclination of the inclined part 38.

The insert 30 may also include one (or more) connection(s) 47 arranged on the annular culture area 34 and designed to cooperate in a removable manner with the interior 14 of the container body 12. The connections 47 may prevent the insert 30 from moving relative to the container body 12 during transport. According to one embodiment, and as shown in the figures, the connections 47 are tabs designed to be removably inserted into a housing 48 on the interior 14 of the container body 12. The housing 48 may be an open recess for removing the insert 30 upward.

The insert 30 may not have the connections 47. Conversely, the insert 30 may have tabs 49 (two or more) arranged on the side edges of the insert 30 with, for example, depressions in the region of the tabs for placing and sliding the insert 30. The tabs 49 are shown in FIG. 4 as dotted lines. The tabs 49 make it possible to reduce the amount of culture medium required for cell culture.

The insert 30 may be made of polypropylene, fluorinated ethylene propylene, or polycarbonate. The insert 30 may be resistant to dimethyl sulfoxide (DMSO).

The container 10 may be used in this way. Initially, the container 10 is delivered with the closure means 18 sealed, and the insert 30 in the interior 14 of the container 10 if the container 10 has such an insert. The insert 30 may contain cells in the working area 36 (i.e., on the working surface 32 if the container does not have an insert, or on the porous base 46 if the container does have an insert) which will be used to culture the cell tissue TC. The container 10 is then connected via the fluidic connections 20 to culture media via the tubes T. There may be one or more culture media which are conveyed into the working area 36 via the fluidic inlet connections 22, sequentially, with (or without) rest times therebetween. The fluidic outlet connection 24 drains the working area 36 between the different instances of conveying culture media. Cell tissue TC culture is therefore carried out in a closed environment with little risk of contamination. Once the cell tissue TC has been created, the container 10 can be cryogenically frozen and transported. When the operator decides to use the cell tissue TC, there are several ways they can recover it. They can open the closure means 18 and recover the cell tissue TC. If the container 10 has the insert, they may then remove the insert through the upper opening 16 thus freed of the closure means 18.

According to one embodiment, and as shown in FIG. 6, the porous base 46 of the insert 30 may be detachable, if necessary via a tongue, such that the cell tissue TC can be extracted from the insert 30 without damaging it. The porous base 46 with the cell tissue TC on top may initially be detached from the insert 30, then the porous base 46 may be removed from the cell tissue TC such that the cell tissue TC can be applied alone, for example, to the patient's skin S.

According to another embodiment, and as shown in FIG. 7, the porous base 46 is detached from the insert 30 and the cell tissue TC remains attached to the insert 30 for some time. The insert 30 is then applied to the patient's skin S in order to place the cell tissue TC on the patient's skin S without manipulating it directly.

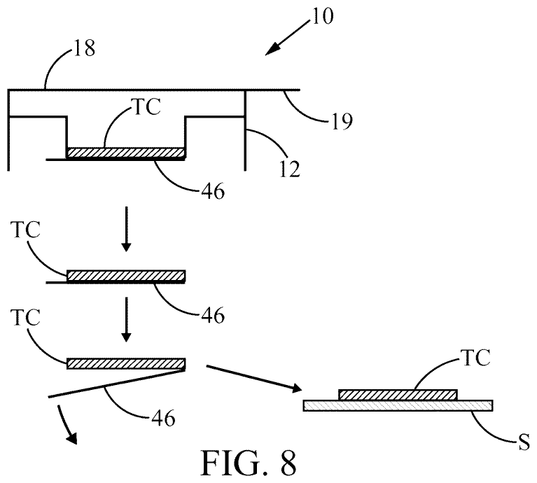

Alternatively, the container 10 could have a removable base for recovering the cell tissue TC, in particular when the container 10 does not have an insert. In relation to FIG. 8, the working surface 32 of the container body 12 is removable and preferably flexible. When the cell tissue TC has been formed and is ready to be used, the working surface 32 is then removed from the container body 12 along with the cell tissue TC. The cell tissue TC is then removed from the working surface 32 and deposited on the patient's skin S.

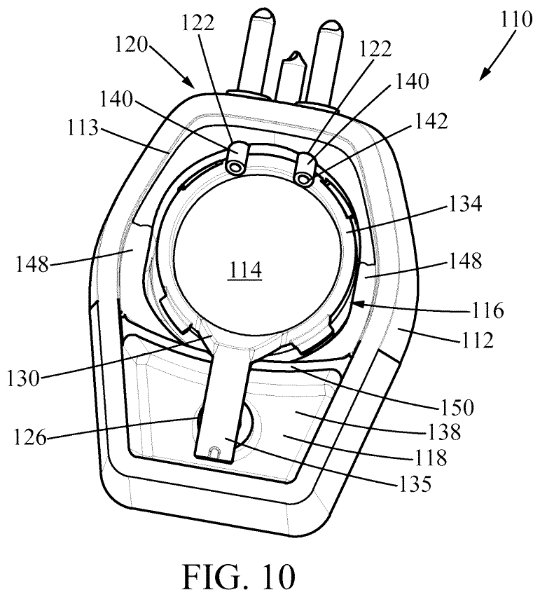

Now with reference to FIG. 10, another embodiment of the container 10 (referred to as the container 110) will be described. The container 110 comprises all the elements and embodiments of the container 10, which will not be described again and for which similar numerical references but in the hundreds will be used.

The container 110 differs from the container 10 in that the container body 112 comprises a partition 150 separates the working area 136 from the filter 126. More specifically, in the embodiment in the figures, the partition 150 is located between the working area 136 and the inclined part 138 in the interior 114 of the container body 112. As such, and as shown in FIG. 10, in this embodiment the part 138 may not be inclined. It is also positioned lower than the part 38 so as to prevent the partition 150 from touching the closure means 118 closing the upper opening 116. Generally, the partition 150 is vertical and does not touch the closure means 118 closing the upper opening 116 of the container body 112. The partition 150 secures the filter and prevents it from becoming wet when the container is being manipulated and during phases of agitation.

1. A cell tissue culture and transport container comprising:
a container body having an accessible interior and an upper opening allowing communication between the interior and an exterior of the container body;
a filter arranged in the container body or on the exterior of the container body, the filter placing the interior in communication with the exterior of the container body, the filter being configured to allow a gas exchange between the interior and the exterior of the container body, while preventing contamination of the cell tissue culture;
a closure means closing the upper opening of the container body, the closure means being sealed or slightly permeable with respect to fluids and gases; and
at least one aseptic fluidic connection through the container body, said at least one aseptic fluidic connection including two fluidic inlet connections for fluids and one fluidic outlet connection for fluids.

2. The container of claim 1, wherein the closure means is disposable, such that once it has been removed from the upper opening, the interior of the container body is accessible through the upper opening.

3. The container of claim 1, wherein the closure means is transparent.

4. The container of claim 1, further comprising a removable insert designed to be arranged in the interior of the container body, the insert being designed to receive a cell tissue (TC) culture.

5. The container of claim 4, wherein the insert can be removed from the container body through the upper opening (16).

6. The container of claim 4, wherein the insert has a culture area and a gripping area connected to the culture area, the gripping area being designed to manipulate the insert relative to the container body.

7. The container of claim 6, wherein the culture area includes an upper opening designed to accommodate the cell tissue (TC) culture.

8. The container of claim 6, wherein the culture area includes a porous base.

9. The container of claim 8, wherein the porous base can be removed from the rest of the insert.

10. The container of claim 8, wherein the porous base is arranged opposite the upper opening when the insert is arranged in the container body.

11. The container of claim 8, wherein the porous base is made of polyester, polyethylene, or polycarbonate.

12. The container of claim 6, wherein the insert includes at least one connection designed to cooperate in a removable manner with the interior of the container body.

13. The container of claim 1, wherein the filter is arranged in an inclined part of the interior of the container body.

14. The container of claim 1, wherein the interior of the container body includes a working area designed to receive the cell tissue (TC) culture and fluid culture media allowing the cell tissue (TC) to grow.

15. The container of claim 1, wherein the body of the container body also comprises a partition separating the working area from the filter.

16. The container of claim 2, further comprising a removable insert designed to be arranged in the interior of the container body, the insert being designed to receive a cell tissue (TC) culture.

17. The container of claim 5, wherein the insert has a culture area and a gripping area connected to the culture area, the gripping area being designed to manipulate the insert relative to the container body.

18. The container of claim 7, wherein the culture area includes a porous base.

19. The container of claim 9, wherein the porous base is arranged opposite the upper opening when the insert is arranged in the container body.

20. The container of claim 7, wherein the insert includes at least one connection designed to cooperate in a removable manner with the interior of the container body.
